(19) United States
(12) Patent Application Publication
(10) Pub. No.: US 2013/0102564 A1
(43) Pub. Date: Apr. 25, 2013

(54) TOPICAL PHARMACEUTICAL COMPOSITION COMPRISING HEPARIN

(71) Applicant: Michele Virno, Roma (IT)

(72) Inventor: Michele Virno, Roma (IT)

(73) Assignee: ADVANCE HOLDINGS LIMITED, Floriana (MT)

(21) Appl. No.: 13/696,735

(22) PCT Filed: May 2, 2011
(86) PCT No.: PCT/EP2011/056931

§ 371 Date: Jan. 9, 2013

Related U.S. Application Data
(60) Provisional application No. 61/347,626, filed on May 24, 2010.

(30) Foreign Application Priority Data
May 7, 2010 (IT) MI2010A 000816

(51) Int. Cl. A61K 9/00

(52) U.S. Cl. CPC A61K 9/0014
USPC 514/56

(57) ABSTRACT

The present invention relates to a topical pharmaceutical composition comprising heparin and to the use thereof for preventing a functional complication of A-V fistulas and A-V grafts in chronic haemodialysis patients.

### FIELD OF THE INVENTION

The present invention relates to a topical pharmaceutical composition comprising heparin and to the use thereof for treating or preventing functional complications of A-V fistulas and grafts in chronic haemodialysis patients.

### BACKGROUND OF THE INVENTION

Heparin is widely used in association to haemodialysis catheters both to prevent catheter occlusion and to prevent complications like peri-catheter thrombosis, acute superficial phlebitis, and the like.

Generally it is proposed to use catheters pretreated with heparin. Alternatively, intermittent flushes and/or continuous infusions of heparin through the catheter are performed (A. G. Randolph et al., BMJ, 316, 969-975 (1998)). Both techniques do not always give satisfactory results.

Therefore, the main concerns with the use of an intravascular catheter remain complications associated with the insertion and residence of these prosthetic materials, such complications including infections and thrombosis of central veins. Thus the catheter use is preferably to provide a temporary vascular access.

A clinical study (Villardell M. et al., Eur. J. Clin. Pharmacol., 54(12), 917-21 (1999)) suggests the use of topical heparin for the treatment of acute superficial phlebitis secondary to indwelling intravenous catheter concluding that topical heparin is safe and effective.

Direct linking of artery and vein, an arteriovenous (A-V) fistula, without use of chronically implanted prosthetic material, has proven to be the best kind of vascular access, but feasible only for subjects whose veins are large enough. The A-V fistula works longer and is also less prone than other types of access to loose its functionality or become infected.

When the creation of an A-V fistula is difficult, an A-V graft (J. A. Akoh, The Journal of Vascular Access, 10, 137-147 (2009)) involving synthetic or biological (autologous, allogeneic or xenogeneic) prosthetic material may be created.

A long-standing A-V fistula or graft may develop functional complications related to a cascade of events starting with neointimal hyperplasia leading to stenosis and subsequently to occlusion of the fistula (D. M. Hentschel, Nephrology Rounds, January 2008, Vol 6, Issue 1; www.nephrologyrounds.org).

Again the main target is to extend the life of the primary patency and the functional or secondary patency (H. J. T. Huijbregts et al., Clin. J. Am. Soc. Nephrol., 3, 714-719 (2008) and L. M. Dember et al., Clin. Trias, 2005; 2; 413)). The maintenance of primary patency is confirmed by the detection of a bruit audible with a stethoscope throughout systole and diastole along the vein at least 8 cm proximal to the AV fistula or graft. The maintenance of functional or secondary patency is confirmed by obtaining a nominal dialysis machine blood flow of at least 300 ml/min.

The usual approach to maintain the fistula patency (and patient's survival) is a systemic treatment of the patient with antiplatelet drugs, like aspirin, ticlodipine, dipyridamole, and clopidogrel (Osborn G, Escofet X, Da Silva A., “Medical adjuvant treatment to increase patency of arteriovenous fistulae and grafts”, Cochrane Database of Systematic Reviews 2008, Issue 4), and intraoperative heparin (H. Ravari, et al., Acta Medica Iranica, 46(5), 379-382 (2008)). These treatments are effective, but strongly increase the haemorrhage risk. Other practices like radiations, local delivery of cell cycle inhibitors (e.g., paclitaxel), angioplasty, surgical interventions, etc., are currently evaluated.

However, an effective, non invasive, practically devoid of side effect treatment applicable without the help of specialized assistance is still lacking.

WO2005/027993 discloses a method for reducing long term vascular access complications associated with a hemodialysis therapy in a patient comprising: a) applying topically to a hemodialysis vascular access site during a hemodialysis session a composition comprising an amount of a vasoconstrictor or coagulant effective for reducing or ceasing post hemodialysis bleeding at the vascular access site; and b) applying compression to the hemodialysis vascular access site for a period of about one to fourteen minutes. While the disclosure of WO2005/027993 generally discusses the problems commonly associated with repeated vascular access, like hyperplasia, thrombosis, hematoma, venous stenosis, arterial stenosis, vascular occlusion, infection, and morbidity, no measures were suggested to treat or prevent functional complications of A-V fistulas or grafts.

Topical products based on heparin sodium are currently available in some European markets.

WO1997/030714 discloses topical pharmaceutical composition comprising heparin in the form of cream, ointment or gel for the treatment of thrombosis, hematomas of any etiology, including posttraumatic hematoma and postcytopenia hematoma, chronic venestasia, and diffuse hematoma patches.

The most diffused products are Lioton™ and Menaven™ in gel form and Viatromb™ and Lipohep™ in liposomal spray form (as described in U.S. Pat. No. 5,958,379).

Topical heparins are widely used for the prevention and treatment of local symptoms associated with peripheral vascular disorders due to their capacity to improve the local microcirculation.

In particular, Lipohep™/Viatromb™ containing 2,400 IU/g of liposomal heparin was shown to be effective as subcutaneous low-molecular-weight heparin in relieving local symptoms of superficial venous thrombosis (SVT), (Grzegorz Górski, M D et al., “Liposomal Heparin Spray: A New Formula in Adjunctive Treatment of Superficial Venous Thrombosis”, Angiology, Vol. 56, No. 1, 9-17 (2005)). The SVT is a condition generally less severe than that represented by fistulas/grafts and its etiopathology is based on completely different grounds.

Liposomal heparin formulations have several disadvantages in terms of process control and process equipment. The preparation of heparin liposomes having a desired particle size requires to proceed stepwise, by preparing the liposomes under stirring with a homogenizer for defined time and controlling the particle size, repeating the operation after addition of each component and continuing the homogenizer stirring if the size is larger than desired. In case the obtained size is smaller, it is practically impossible to reverse it so the batch must be wasted. To complicate the situation further, phospholipids of adequate quality degree are rather expensive and, being of natural origin, are characterized by an intrinsic variability.

All these aspects contribute to render difficult and expensive the production, requiring several in-process controls with high and unavoidable risk to obtain batches out of specification.

### SUMMARY OF THE INVENTION

The Applicant has had the idea of using topical heparins to prevent functional complications and to maintain the patency of A-V fistulas and grafts. The main functional complication is the neointimal hyperplasia leading to stenosis which may reduce the patency up to the occlusion of the fistula requiring surgical intervention or the creation of a new fistula.

The Applicant has surprisingly found that the regular application of topical heparin to the area of the A-V fistula was able to maintain the patency and to prevent stenosis and/or occlusions of A-V fistulas with an efficacy at least comparable to that of antiplatelet agents without interfering with the coagulation properties of the circulating blood.

The Applicant has also found a new composition for the topical application of heparin represented by a solution of heparin and at least one polyoxyalkylene ester of a hydroxy fatty acid.

The Applicant has further found that the new composition for the topical application of heparin of the present invention shows several distinct advantages with respect to known heparin formulation in the form of gel or liposomal spray.

The composition of the present invention appears as a clear uncolored solution which can be easily filtered before repartition.

The composition of the present invention is much easier prepared since the quality of the product is substantially independent by the stirring speed and normal equipment can be used without need of a homogenizer.

The composition of the present invention does not require in-process controls of particle size.

The polyoxyalkylene ester of a hydroxy fatty acid employed in the composition of the present invention is commercially available at low cost and pharmaceutical quality (e.g., macrogol 15 hydroxystearate), and it does not require special purification and qualification to be used as drug excipient.

Further, therapeutic efficacy is at least the same, sometimes with advantages.

### DETAILED DESCRIPTION OF THE INVENTION

Accordingly, the present invention relates to a new pharmaceutical formulation for the topical application of heparin represented by a solution of heparin and at least one polyoxyalkylene ester of a hydroxy fatty acid.

Advantageously, said solution of heparin and at least one polyoxyalkylene ester of a hydroxy fatty acid is a solution in water, at least one alcohol or a mixture thereof. Preferably, said at least one alcohol is selected from the group comprising pharmaceutically acceptable alcohols, such as, for example, ethanol, 1-propanol, 2-propanol, and mixtures thereof. Preferably, said solution of heparin and at least one polyoxyalkylene ester of a hydroxy fatty acid is a solution in water.

For the purposes of the present invention and the claims appended herein, the term “topical application” means a skin application, preferably with a substantially local effect, and essentially devoid of systemic effect.

As used herein, the term “heparin” refers to any type of pharmaceutically-acceptable heparin, heparinate, or heparinoid.

Consequently, as used herein, the term “heparin” includes complexed and uncomplexed heparin, heparin salts such as sodium heparin, potassium heparin, calcium heparin and magnesium heparin, heparin esters, and heparinic acids.

Such heparin compounds are widely available from a large number of commercial sources. For instance, calcium heparin is sold under the tradenames CALCIPARINE and ECASOLV, magnesium heparin is available under the tradename CUTHEPARINE, and sodium heparin is available under many tradenames including HEPRINAR and HEPSAL. Commercially available heparin is isolated from bovine lung or pork intestinal mucosa and generally has a molecular weight between 6 and 30 kD.

Preferably, said at least one polyoxyalkylene ester of a hydroxy fatty acid is obtained from the esterification of a hydroxy fatty acid having from 8 to 30 carbon atoms, preferably from 14 to 24 carbon atoms, with a polyoxyalkylene having a molecular weight ranging from 200 to 6,000, preferably from 400 to 1,500.

Advantageously, said hydroxy fatty acids are selected from the group comprising saturated chains, such as hydroxycaprylic acid, hydroxycapric acid, hydroxylauric acid, hydroxymyristic acid, hydroxypalmitic acid, hydroxystearic acid, hydroxyarachidic acid, hydroxybehenic acid, hydroxylignoceric acid, and unsaturated chains, such as hydroxymyristoleic acid, hydroxypalmitoleic acid, hydroxyoleic acid, hydroxylinoleic acid, hydroxylinolenic acid, hydroxyarachidonic acid, hydroxyeicosapentaenoic acid, hydroxyerucic acid, and hydroxydocosahexaenoic acid.

Particularly useful hydroxy fatty acids are selected from the group of saturated hydroxy fatty acids comprising hydroxylauric acid, hydroxymyristic acid, hydroxypalmitic acid, hydroxystearic acid, and hydroxyarachidic acid. The Applicant has found that the use of saturated hydroxy fatty acids is preferable, because the presence of unsaturation in the fatty acid chains can favor the degradation by oxidation and reduce the lifetime of the pharmaceutical formulation. A particularly preferred hydroxy fatty acid is hydroxystearic acid.

Advantageously, said polyoxyalkylene is selected from the group comprising polyethylene glycol 200 (PEG 200), polyethylene glycol 300 (PEG 300), polyethylene glycol 400 (PEG 400), polyethylene glycol 600 (PEG 600), polyethylene glycol 660 (PEG 660), polyethylene glycol 1000 (PEG 1000), polyethylene glycol 1500 (PEG 1500), polyethylene glycol 3000 (PEG 3000), polyethylene glycol 3350 (PEG 3350), polyethylene glycol 4000 (PEG 4000), polyethylene glycol 6000 (PEG 6000), and mixtures thereof.

According to a preferred embodiment, said polyoxyalkylene comprises polyethylene glycol 400 (PEG 400), polyethylene glycol 600 (PEG 600), polyethylene glycol 660 (PEG 660), polyethylene glycol 1000 (PEG 1000), polyethylene glycol 1500 (PEG 1500), and mixtures thereof.

According to a preferred embodiment of the present invention said at least one polyoxyalkylene ester of a hydroxy fatty acid is selected from the group of Solutol™ HS 15 (polyethylene glycol 660 hydroxy stearate—Ph. Eur. Name: Macrogol 15 Hydroxystearate), a polyglycol ester of polyethylene glycol and 12-hydroxystearic acid, and mixtures thereof.

Solutol HS 15 is a polyethylene glycol 660 hydroxystearate manufactured by BASF (Parsippany, N.J.). Apart from free polyethylene glycol and its monoesters, diesters are also detectable. According to the manufacturer, a typical lot of Solutol HS 15 contains approximately 30% free polyethylene glycol and 70% polyethylene glycol esters.

The concentration of said at least one polyoxyalkylene ester of a hydroxy fatty acid in the pharmaceutical formulation of the present invention is preferably from 1% to 20% (w/v), more preferably from 2% to 15% (w/v), and most preferably from 5% to 15% (w/v). Advantageously, the concentration of the polyoxyalkylene hydroxy fatty acid ester is from about 5% to about 10% (w/v).

The pharmaceutical formulation of the present invention is an aqueous solution. Advantageously, the pharmaceutical formulation of the present invention may also comprise at least one alcohol.

Preferably, said at least one alcohol is selected from the group comprising pharmaceutically acceptable alcohols, such as for example, ethanol, 1-propanol, 2-propanol, and mixture thereof.

The concentration of said at least one alcohol in the pharmaceutical formulation of the present invention is preferably from 1% to 40% (w/v), more preferably from 2% to 30% (w/v), and most preferably from 5% to 25% (w/v).

The pH of the pharmaceutical formulation of the present invention is preferably ranging from 5 to 8, more preferably from 5.5 to 7.5. Advantageously, the pH of the pharmaceutical formulation of the present invention is ranging from 6 to 7.

The pharmaceutical formulation of the present invention may further comprise several additives generally known and used in the art. Such non-essential additives of the pharmaceutical formulation according to the invention are, for example, stabilizers, antioxidants, pH correctors, buffers, surfactants, colorants and/or perfumes.

The pharmaceutical formulation according to the present invention can be formulated into a preparation form which is commonly employed as a preparation form for topical application.

Advantageously useful preparation forms include various solutions, sprays, foams, cataplasm plasters, and the like. Topical preparations in the form of solution and spray are particularly preferred.

The pharmaceutical formulation of the present invention is useful for treating or preventing functional complications of A-V fistulas and A-V grafts in chronic haemodialysis patients.

Advantageously, the pharmaceutical formulation of the present invention is useful for treating or preventing neointimal hyperplasia leading to stenosis reducing and/or occluding said A-V fistulas and said A-V grafts.

The pharmaceutical formulation of the present invention is then useful for the treatment and prevention of those phenomena reducing the primary and/or secondary patency of A-V fistulas and A-V grafts in chronic haemodialysis patients.

Advantageously, the pharmaceutical formulation of the present invention can be used in combination with antiplatelet drugs and systemic heparin for treating or preventing functional complications of A-V fistulas and A-V grafts in chronic haemodialysis patients. The use of the pharmaceutical formulation of the present invention in combination with antiplatelet drugs and/or systemic heparin allows to reduce the dosage of such antiplatelet drugs and/or systemic heparin, and then to reduce the adverse effect thereof.

The following examples further illustrate the invention, without limiting it.

### EXAMPLES

### Example 1

### Preparation of Heparin Liposomal Formulation (2,400 IU/ml)

In an adequate container were introduced water (ml 315) and NAT 8539 (g 87). NAT 8539® (Phospholipid GmbH, Cologne, Germany) is a mixture of ethanol (25% by weight) and Phospholipon 80 (75% by weight), the latter being a soy bean lipid extract comprising phosphatidylcholine (76% by weight) and minor amounts of lyso-phosphatidylcholine (up to 6%), cephaline (up to 4%) and phosphatidic acid.

The mixture was stirred with a homogenizer at 30 rpm at room temperature for 30 min. Ethanol 96% (ml 111) was added and stirring was continued for 30 min.

The particle size present in the mixture was periodically controlled, and the stirring was continued until reaching the desired average particle size of about 150 nm. Stirring operation must be carefully conducted to avoid the formation of smaller than desired particle size, due to the difficulty, and even impossibility, of returning to a larger size.

In another adequate container water (120 ml) and sodium heparin (10.4 g; 150 IU/ml) were introduced and stirred until to complete dissolution.

The heparin solution was slowly transferred while stirring into the first container (containing water plus NAT 8539). Stirring was continued 30 min. The particle size present in the mixture was periodically controlled, and the stirring was continued until reaching the desired average particle size.

When the desired average particle size was reached, a water solution (29 ml) containing 3.38 g of potassium dihydrogen phosphate and 0.26 g of sodium hydroxide (pH of buffer solution: 6.6) was added under stirring. The particle size present in the mixture was periodically controlled, and the stirring was continued until reaching the desired average particle size.

When the desired average particle size was reached, the volume was brought to 650 ml with water and the pH was finally checked to verify that its value was between 6 and 7.

The final liposomial solution was poured in 30 ml bottles (filled up to 25 ml) and closed with a spray metered pump able to supply 200 μl per puff. The preparation is an analogue of the commercial preparation of liposomial heparin sold under the trademark Viatromb™.

### Example 2

### Preparation of Heparin Solution (Solution A—2,400 IU/ml)

In an appropriate container water (400 ml) and sodium heparin (10.4 g: 150 IU/mg) were introduced. The mixture was stirred up to complete dissolution and, under stirring, macrogol 15 hydroxy stearate (65 g) was added.

After complete dissolution, ethanol 96% (ml 140) and a water solution (29 ml) containing 3.38 g of potassium dihydrogen phosphate and 0.26 g of sodium hydroxide (pH of buffer solution: 6.6) was added under stirring. The volume was brought to 650 ml with water and the pH was finally checked to verify that its value was between 6 and 7.

The final solution A was poured in 30 ml bottles (filled up to 25 ml) and closed with a spray metered pump able to supply 200 μl per puff.

### Example 3

### Preparation of Heparin Solution (Solution B—2,400 IU/ml)

The same procedure of preparation A was followed using the same volume of isopropanol in place of ethanol.

### Example 4

### Preparation of Heparin Solution (Solution C—2,400 IU/ml)

The same procedure of preparation A was followed using the same volume of water in place of ethanol.

Effect on A-V Fistula Patency Over Time

Sixty patients of different age and sex, to whom has just been created a A-V fistula for dialysis, were randomly divided in three groups. Each group was attributed a different treatment, namely a topical treatment with Viatromb™, a topical treatment with Solution A and a systemic treatment with antiplatelet drugs.

Both comparison solution Viatromb™ and invention solution A were applied topically in the area of the fistula by means of three puffs twice a day.

The antiplatelet treatment was selected by the study director among aspirin, ticlodipine, dipyridamole and clopidogrel according to the characteristics of the patient. The standard regimen used for this type of protocols was followed.

The results were summarized in the following Table 1.

TABLE 1

| Time | Solution | Solution | Antiplatelet |
|---|---|---|---|
| (months) | Viatromb ™ | A | drug |
| 1 | 19/20 | 19/20 | 18/20 |
| 3 | 18/20 | 19/20 | 17/20 |
| 6 | 18/20 | 19/20 | 16/20 |

The reduction of the number of patients during time was due to the therapy interruption due to different causes, such as fistula occlusion and side effects due to the treatment with antiplatelet drugs. The results have been expressed as number of patients with a working A-V fistula, i.e., with a maintained secondary patency, out of the total number of treated patients at the showed time.

The results have confirmed the efficacy of antiplatelet therapy, but topical heparin formulations were definitely more effective in maintaining the patency. The topical treatment further showed a better tolerability than the systemic treatment as it has not caused any adverse effect. The B and C solutions have shown properties similar to those of solution A.

1. Topical pharmaceutical formulation comprising a solution of heparin and at least one polyoxyalkylene ester of a hydroxy fatty acid.

2. Pharmaceutical formulation for the topical application of heparin, wherein said formulation is a solution of heparin and at least one polyoxyalkylene ester of a hydroxy fatty acid.

3. Pharmaceutical formulation according to claim 1 or 2, wherein said formulation is an aqueous solution.

4. Pharmaceutical formulation according to claim 3, wherein said aqueous solution further comprises at least one alcohol.

5. Pharmaceutical formulation according to claim 4, wherein said alcohol is selected from the group comprising pharmaceutically acceptable alcohols, preferably ethanol, 1-propanol, 2-propanol, and mixture thereof.

6. Pharmaceutical formulation according to any one of preceding claims, wherein said at least one polyoxyalkylene ester of a hydroxy fatty acid is selected from the group consisting of esters of a hydroxy fatty acid having from 8 to 30 carbon atoms, preferably from 14 to 24 carbon atoms, with a polyoxyalkylene having a molecular weight ranging from 200 to 6,000, preferably from 400 to 1,500.

7. Pharmaceutical formulation according to claim 6, wherein said at least one polyoxyalkylene ester of a hydroxy fatty acid is a polyglycol ester of polyethylene glycol and 12-hydroxystearic acid (Macrogol 15 hydroxystearate).

8. Pharmaceutical formulation according to any one of preceding claims, for the treatment or prevention of a functional complication of A-V fistulas and A-V grafts in chronic haemodialysis patients.

9. Pharmaceutical formulation according to claim 8, wherein said functional complication is the neointimal hyperplasia leading to stenosis reducing and/or occluding said A-V fistulas and A-V grafts.

10. Use of a heparin for the preparation of a topical pharmaceutical formulation for the treatment or prevention of a functional complication of A-V fistulas and A-V grafts in chronic haemodialysis patients.

11. Use of a heparin for the preparation of a topical pharmaceutical formulation for the treatment or prevention of phenomena reducing the primary and/or secondary patency of A-V fistulas and A-V grafts in chronic haemodialysis patients.

12. Use of a topical pharmaceutical formulation comprising heparin in combination with a systemic pharmaceutical formulation comprising heparin for the treatment or prevention of a functional complication of A-V fistulas and A-V grafts in chronic haemodialysis patients.

13. Use of a topical pharmaceutical formulation comprising heparin in combination with a systemic pharmaceutical formulation comprising antiplatelet drugs for the treatment or prevention of a functional complication of A-V fistulas and A-V grafts in chronic haemodialysis patients.
